(19) United States
(12) Patent Application Publication
(10) Pub. No.: US 2024/0138728 A1
(43) Pub. Date: May 2, 2024

(54) WEARABLE VITAL SIGNS MONITOR WITH SKIN TONE DETECTION

(71) Applicant: BIOINTELLISENSE, INC., Golden, CO (US)

(72) Inventor: Thomas Dietiker, Palos Verdes Estates, CA (US)

(21) Appl. No.: 18/499,977

(22) Filed: Nov. 1, 2023

Related U.S. Application Data
(60) Provisional application No. 63/382,106, filed on Nov. 2, 2022.

(51) Int. Cl. A61B 5/1455; A61B 5/00; G01N 21/25

(52) U.S. Cl. CPC A61B 5/14552; A61B 5/0075; A61B 5/441; A61B 5/7203; A61B 5/7246; A61B 5/7271; G01N 21/25; A61B 2560/02; A61B 2560/0462; A61B 2562/0238

(57) ABSTRACT

In an example embodiment, an optical vital signs monitor includes a light source, a spectral sensor, a processor, and a non-transitory computer readable medium that includes instructions executable by the processor to perform or control performance of operations. The operations include illuminating, via the light source, a body of a subject. The operations include receiving, via the spectral sensor, a resultant signal from the body of the subject. The operations include determining a skin tone of the subject from the received resultant signal.

### CROSS-REFERENCE TO RELATED APPLICATION

This application claims the benefit of and priority to U.S. Provisional Patent Application No. 63/382,106, filed Nov. 2, 2022. The 63/382,106 application is incorporated herein by reference in its entirety for all purposes.

### FIELD

The embodiments discussed herein are related to a wearable vital signs monitor with skin tone detection.

### BACKGROUND

Unless otherwise indicated herein, the materials described herein are not prior art to the claims in the present application and are not admitted to be prior art by inclusion in this section.

Monochromatic light sources, often light-emitting diodes (LEDs), are utilized in vital sign monitors to obtain non-invasive measurements of blood components or for use in pulse oximetry. Typically, to obtain accurate measurements utilizing a monochromatic light source, the wavelength of the monochromatic light source must be accurately controlled to determine the amount of monochromatic light absorbed and reflected. Thus, LEDs or other light sources used with conventional processes must meet wavelength tolerances for a respective application, and/or additional signal processing techniques for the associated wavelength.

Moreover, utilizing a conventional monochromatic light source, significant variations arise from the movement of a user, skin tone, and the part of the body from which measurements are obtained, and must be eliminated or reduced. Thus, conventional monochromatic vital sign monitors are limited in implementation and use within wearable devices.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, an optical vital signs monitor includes a light source, a spectral sensor, a processor, and a non-transitory computer readable medium that includes instructions executable by the processor to perform or control performance of operations. The operations include illuminating, via the light source, a body of a subject. The operations include receiving, via the spectral sensor, a resultant signal from the body of the subject. The operations include determining a skin tone of the subject from the received resultant signal.

In another example embodiment, a method includes illuminating, via a light source of an optical vital signs monitor, a body of a subject. The method includes receiving, via a spectral sensor of the optical vital signs monitor, a resultant signal from the body of the subject. The method includes determining a skin tone of the subject from the received resultant signal.

Additional features and advantages of these embodiments will be set forth in the description which follows, and in part will be obvious from the description, or may be learned by the practice of the embodiments. The features and advantages of these embodiments may be realized and obtained by means of the instruments and combinations particularly pointed out in the appended claims. These and other features of the present embodiments will become more fully apparent from the following description and appended claims or may be learned by the practice of the embodiments as set forth hereinafter.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a block diagram illustrating a system for a wearable vital signs monitor;

FIG. 2 is a flow diagram of a method for determining SpO2 based on a polychromatic light source;

FIG. 3 is a lookup table for obtaining vital signs measurements in a wearable vital signs monitor;

FIG. 4 is a flow diagram of a method of utilizing a wearable vital signs monitor; and

FIG. 5 is a schematic block diagram of a computer system for a wearable vital signs monitor,

all arranged in accordance with at least one embodiment described herein.

### DETAILED DESCRIPTION OF SOME EXAMPLE EMBODIMENTS

The following detailed description illustrates a few example embodiments in further detail to enable one of skill in the art to practice such embodiments. The described examples are provided for illustrative purposes and are not intended to limit the scope of the invention.

In the following description, for the purposes of explanation, numerous specific details are set forth to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Certain embodiments provide tools and techniques for wearable vital signs monitoring with skin tone detection and compensation. The tools provided by various embodiments include, without limitation, systems, apparatuses, methods, and/or software products. Merely by way of example, a method might comprise one or more procedures, any or all of which are executed by a computer system. Correspondingly, an embodiment might provide a computer system configured with instructions to perform one or more procedures in accordance with methods provided by various other embodiments. Similarly, a computer program might comprise a set of instructions that are executable by a computer system (and/or a processor therein) to perform or control such operations. In many cases, such software programs are encoded on physical, tangible and/or non-transitory computer readable media (such as, to name but a few examples, optical media, magnetic media, and/or the like).

In an aspect, a system for a wearable vital signs monitor is provided. The system may be configured to be worn by a user and determine the vital signs of the user. The measurement of vital signs may include, without limitation, the measurement of blood oxygen saturation (SpO2), heartrate, blood components, and other physiological parameters. In further embodiments, the system may provide monitoring and telemedicine functionality to facilitate remote diagnosis, assessment, and treatment by a healthcare provider. The system may include a remote device coupled to a communications network, and a wearable device in communication with the remote device via the communications network, the wearable device configured to be in contact with a body of a subject and determine vital signals of a subject. The wearable device further includes a polychromatic or broad spectrum light source, a spectral sensor, a processor, and a non-transitory computer readable medium comprising instructions executable by the processor to perform or control performance of operations. The operations may include illuminating, via the polychromatic light source, a perfused tissue of the body of the subject; receiving, via the spectral sensor, a resultant signal from the body of the subject; and determining a skin tone of the subject from the received resultant signal. In some embodiments, the wearable device may generate physiological measurements of the subject and may adjust or correct the physiological measurements based on the determined skin tone.

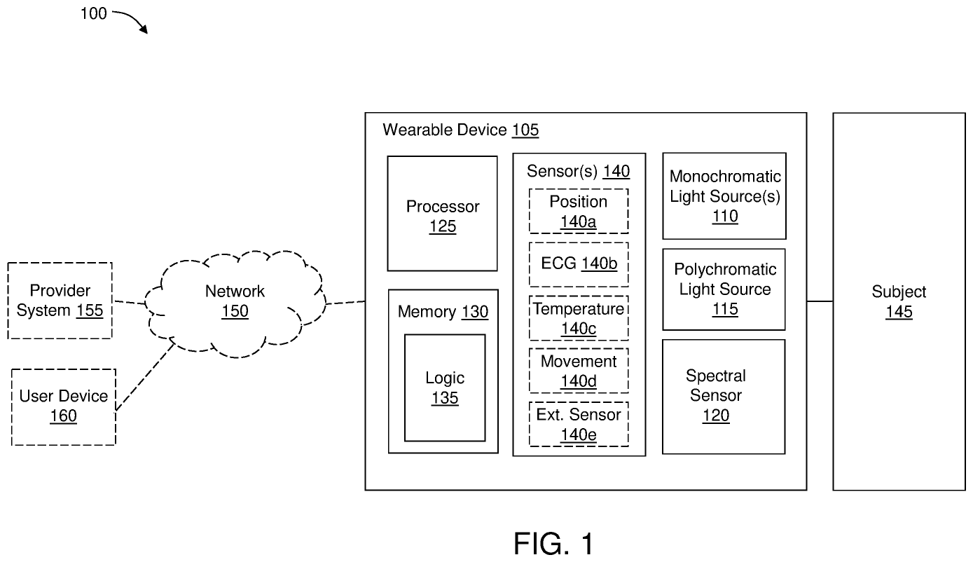

FIG. 1 is a block diagram of a system 100 for a wearable vital signs monitor, in accordance with at least one embodiment described herein. The system 100 may include a wearable device 105 including one or more monochromatic light sources 110, such as red and infrared (IR) LEDs, a polychromatic light source 115, such as a white LED, a photodetector such as spectral sensor 120, sensors 140, a processor 125, system memory 130 and logic 135. The system may further include a network 150, and remote systems such as a provider system 155 or user device 160.

Sensors 140 of the wearable device 105 are optional and may include, without limitation, a position sensor 140a, electrocardiogram (ECG) sensor 140b, temperature sensor 140c, movement sensor 140d, and external sensor 140e. The optional sensors 140 may further include, without limitation, gyroscopes, accelerometer, blood pressure sensors and/or monitors, blood sugar monitors, weight sensors (e.g., scales, load sensors, etc.), magnetometer, camera, global positioning system (GPS) receiver, microphone, Bluetooth sensor (e.g., transmitter, receiver, positional sensor, etc.), or IR sensor (e.g., transmitter, receiver, positional sensor, etc.).

The components of the system 100 are schematically illustrated in FIG. 1, and a variety of hardware configurations are possible in accordance with various embodiments.

In various embodiments, the wearable device 105 may be coupled to a subject 145. The wearable device 105 may be positioned such that the one or more monochromatic light sources 110, polychromatic light source 115, and spectral sensor 120 are in contact or close proximity with the skin of the subject 145. Thus, in various embodiments, the one or more monochromatic light sources 110, polychromatic light source 115, and spectral sensor 120 may be operably coupled to the subject 145. The wearable device 105 may further be coupled to the provider system 155 and/or another remote device, such as the user device 160, via the network 150. Accordingly, in some embodiments, the wearable device 105 may further include a communications subsystem (not illustrated in FIG. 1) configured to communicate over the network 150.

In various embodiments, the processor 125, memory 130 and associated logic 135, one or more monochromatic light sources 110, polychromatic light source 115, spectral sensor 120, and sensors 140 may be part of a vital signs monitor assembly of the wearable device 105. An example of a vital signs monitor assembly may include a self-calibrating sensor system, as described in U.S. Pat. No. 10,485,463 (the '463 patent) issued Nov. 26, 2019 entitled “System and Method for a Non-invasive Medical Sensor.” The disclosure of the '463 patent is incorporated herein by reference in its entirety for all purposes.

In various embodiments, the one or more monochromatic light sources 110 may include one or more light sources configured to generate light in a respective wavelength. For example, the one or more monochromatic light sources 110 may include, without limitation, a red LED, an IR LED, or both red and IR LEDs. Although described as generating light at a singular wavelength, in some practical applications, the one or more monochromatic light sources 110 may generate light over a narrow range of wavelengths (e.g., narrow bandwidth) corresponding to a single color of light. For example, the one or more monochromatic light sources 110 may include a red LED configured to emit light at a wavelength of 660 nm, and in some embodiments, within a range of +/−12 nm. In some examples, the one or more monochromatic light sources 110 may include an IR LED configured to emit light at a wavelength of 880 nm, within a range of +/−25 nm. In various embodiments, the polychromatic light source 115 may include any light source, or combination of multiple light sources, configured to generate light in more than one wavelength (e.g., two or more wavelengths, three or more wavelengths, etc.). In various embodiments, the polychromatic light source 115 may include, without limitation, one or more broadband light sources, such as a white LED, a combination of two or more monochromatic light sources (e.g., a red, green, and blue (RGB) LEDs), or other combinations of polychromatic and/or monochromatic light sources (e.g., a combination of white LEDs and RGB LEDs). As will be appreciated by those skilled in the art, unlike the one or more monochromatic light sources 110, a polychromatic light source 115 may generate light over a broad range of wavelengths (e.g., broad bandwidth and/or full-spectrum), or alternatively, generate light over multiple ranges of wavelengths corresponding to two or more colors of light. For example, a polychromatic light source 115 may include a light source that emits light over a range of wavelengths in the visible spectrum, ultraviolet (UV), near-IR, and IR. In one example, a polychromatic light source 115 may include a light source that emits light in the range of 200 nm to 2000 nm, or one or more portions of the spectrum from 200 nm to 2000 nm.

Suitable position sensors 140a may include, without limitation, a gyroscope, Bluetooth-based positional sensor, an accelerometer, or a combination of sensors. Temperature sensors 140c may include, without limitation, a thermometer (e.g., resistance thermometer, IR thermometer), thermistor, thermocouple, or other suitable temperature sensors as known to those skilled in the art. Movement sensors 140d may include motion detectors (e.g., acoustic, IR, microwave, ultrasonic, video, etc.), accelerometers, among other sensors suitable for detecting movement of the wearable device. The external sensor 140e may include any further external sensor that may be coupled to the wearable device 105. For example, the external sensor 140e may include, without limitation, an additional ECG sensor with an extended lead, a camera, microphone, or any other sensor configured to monitor the subject 145. Accordingly, in various embodiments, the wearable device 105 may further include an interface configured to allow the external sensor 140e to be operatively coupled to wearable device 105.

Accordingly, the wearable device 105 may, in some embodiments, include an on-board computer device. For example, the processor 125, memory 130, and logic 135 may be implemented, for example, as part of a programmable logic controller (PLC), single board computer, field programmable gate arrays (FPGA), application specific integrated circuits (ASIC), or a system on a chip (SoC). In various embodiments, the on-board computer, including the processor 125, memory 130, and logic 135, may be configured to perform signal processing and analysis based on signals received from the spectral sensor 120 and/or one or more sensors 140. In further embodiments, at least part of the signal processing and analysis of the signals from the spectral sensor 120 and/or the one or more sensors 140 may be performed by an external computer device, such as one or more desktop computer systems, server computers, and/or dedicated custom hardware appliances, with which the wearable device 105 may be configured to communicate.

In some examples, the wearable device 105 may include various types of personal electronic devices and “smart” clothing, in which clothing may be integrated with a vital signs monitoring assembly (e.g., including the one or more monochromatic light sources 110, polychromatic light source 115, spectral sensor 120, and one or more sensors 140). For example, suitable wearable devices 105 may include, without limitation, a smart watch, smart glasses, a temporarily attachable device, an implantable device, shirts, headwear including headbands and hats, footwear including socks and shoes, pants, undergarments, helmets, wristband devices such as fitness trackers, necklaces, rings, and bracelets, among a growing number of wearable devices which may be held, carried, or worn in close proximity to the body of the subject 145. For example, a wrist band device may be configured to be secured to the wrist of the subject 145. The wrist band device may be configured such that when it is worn by the subject 145, one or more of the polychromatic light source 115, such as a white LED, one or more monochromatic light sources 110, such as a red and IR LED, and the spectral sensor 120 are operatively coupled to the wrist of the subject 145. Similarly, in some embodiments, the wearable device 105 may be a glove configured to cover all or part of the hand of the subject 145. The glove may be configured such that when it is worn by the subject 145, one or more of the polychromatic light source 115, one or more monochromatic light sources 110, and the spectral sensor 120 is operatively coupled to the hand of the subject 145. In some embodiments, the wearable device 105 may be a headband device configured such that when it is worn by the subject 145, one or more of the polychromatic light source 115, one or more monochromatic light sources 110, and spectral sensor 120 is operatively coupled to a forehead of the subject 145.

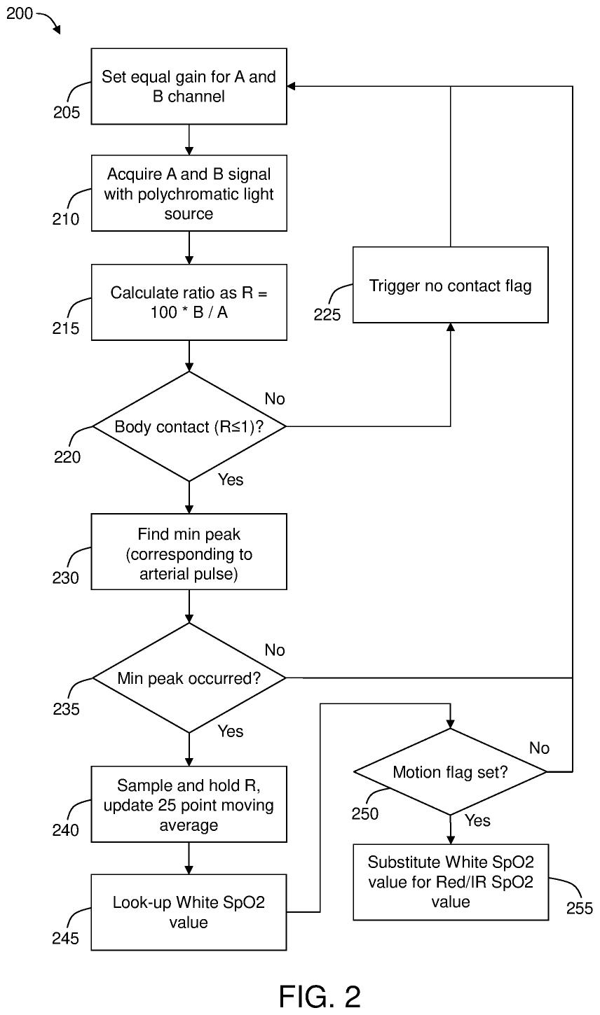

In some embodiments, the wearable device 105 may be a temporarily attachable device configured to attach to a subject 145. Attachment may be achieved, for example, via adhesive. The temporarily attachable device may be configured to be attached to the chest, back, or any other area of the subject's 145 body sufficiently large to accommodate the temporarily attachable device. An example of one such embodiment is described in greater detail below, with respect to FIG. 2.

In various embodiments, the components of the wearable device 105 may be arranged according to a part of the subject 145 to which the wearable device 105 is to be worn over and/or attached, and the specific form factor of the wearable device 105 to accommodate the respective part of the subject 145. For example, in one configuration of a glove, the polychromatic light source 115 and/or one or more monochromatic light sources 110 may be adjacently positioned to the spectral sensor 120 within a threshold proximity of each other. In various embodiments, the threshold proximity may be a distance from the spectral sensor 120 that an illuminated area of the perfused tissue of the hand may be detectable by the spectral sensor 120. Alternatively, using the same example, in some embodiments, the polychromatic light source 115 and one or more monochromatic light sources 110 may be positioned on an opposite side of a finger from the spectral sensor.

In various embodiments, the wearable device 105 may be configured to modify an algorithm for determining the vital signs based, at least in part, on the skin tone of the subject 145.

In various embodiments, the wearable device 105 may further include logic configured to control various functions of the wearable device 105. The logic may be implemented as hardware, software, or both hardware and software. For example, in some embodiments, logic may include a calibration circuit as described in the '463 patent. In some embodiments, the logic may include a set of instructions executable by the hardware of the wearable device 105, such as the processor 125, and may be stored in system memory 130 or other storage device. Thus, the logic may, at least partially, be included as part of a software program or application executable by the processor 125 of the wearable device 105. In other embodiments, the logic may include dedicated hardware, which may also include software. Thus, in some examples, the logic may include, without limitation, an embedded system within the mobile device, such as a field programmable gate array (FPGA), system on a chip (SoC), or application specific integrated circuit (ASIC).

Accordingly, in various embodiments, the wearable device 105 may be configured to measure the vital signs of a subject 145 and generate a corresponding physiological measurement. As previously described, vital signs or corresponding physiological measurements may include, without limitation, a blood oxygen saturation (e.g., peripheral capillary oxygen saturation (SpO2)), heart rate, blood components (e.g., hemoglobin count and/or concentration, carbon monoxide concentration, etc.), or other physiological parameters (e.g., pulse, heart rate, etc.). In various embodiments, the wearable device 105 may be configured to emit light of at least two wavelengths towards perfused human tissue at various sites of the body of the subject 145. In further embodiments, one or more monochromatic light sources 110, such as red and IR LEDs, may be used by the wearable device 105 to illuminate the body of the subject 145. In still further embodiments, a combination of the one or more monochromatic light sources 110 and polychromatic light source 115 may be used by the wearable device 105 to illuminate the body of the subject 145. In some embodiments, the wearable device 105 may, via an on-board computer, such as an on-board controller including the processor 125 and/or memory 130, be configured to cause polychromatic light source 115, such as a white LED, to illuminate the body of the subject 145. For example, the on-board controller, such as processor 125 and memory 130, may include driver circuitry and/or logic for driving the polychromatic light source 115 and/or one or more monochromatic light sources 110.

In various embodiments, after the tissue has been illuminated by a source signal from the polychromatic light source 115, one or more monochromatic light sources 110, or both polychromatic light source 115 and one or more monochromatic light sources 110, the wearable device 105 may be configured to receive a reflected signal from the body of the subject 145, or in alternative embodiments, a transmitted signal through the body of the subject 145. For ease of description, both transmitted and reflected signals will be referred to as the reflected signal in this description, or alternatively “the resultant signal.” The resultant signal may result from the source signal (e.g., incident light) being reflected by (or transmitted through) the body of the subject 145. For example, in various embodiments, the body of the subject 145 may, at least partially, absorb one or more component wavelengths of the source signal. Thus, the differences in wavelengths between the source signal and the resultant signal may indicate different characteristics of the perfused tissue (e.g., fluids in the tissue).

Accordingly, the wearable device 105 may be configured to receive the resultant signal from the body of the subject 145 via one or more photodetectors, such as the spectral sensor 120. In various embodiments, the spectral sensor 120 may include one or more photodetectors configured to measure light power at one or a range of wavelengths. Accordingly, in various embodiments, the wearable device 105 may be configured to determine spectral properties of the reflected signal based on inputs from the spectral sensor 120. For example, the determination of spectral properties may, in some embodiments, include determination of a transmission spectrum and/or a spectral power distribution.

In various embodiments, based on the spectral properties of the resultant signal, the wearable device 105 may be configured to determine one or more vital signs of the subject 145. For example, in some embodiments, the vital signs monitoring assembly of the wearable device 105 may be configured to cause the polychromatic light source 115 to illuminate the tissue of a subject 145 with a broad-spectrum source signal. The resultant signal may be received by the spectral sensor 120. The received resultant signal may then further be processed, for example, by the processor 125, to determine a transmission spectrum and/or spectral power distribution of the resultant signal, indicative of transmission wavelengths with the most optical intensity and/or optical power.

In some embodiments, using a polychromatic light source 115, skin tone of the subject 145 may be detected and corresponding physiological measurements may be corrected based on the subject's skin tone level. For example, a resultant signal from illumination of the subject 145 with the polychromatic light source 115 may be used to determine the subject's skin tone level and then one or more resultant signals from illumination of the subject 145 with one or more of the polychromatic light source 115 or the monochromatic light sources 110 may be used to generate physiological measurements which may be adjusted or corrected based on the skin tone level. Such processes are described in greater detail below.

In some embodiments, one or more of the resultant signals may be processed to obtain a pulse waveform proportional to the arterial and venous pulse of a body. For example, in some embodiments, the pulse waveform may be determined based on an absorption and/or reflectance of various wavelengths of the source signal by the tissue of the subject 145 (e.g., comparing the source signal with the resultant signal), based on the spectral transmission and/or absorption properties of oxygenated (arterial) bhO₂ and deoxygenated (venous) hemoglobin (Hgb) molecules. Additional algorithms for determining vital signs are discussed in further detail in the '463 patent.

In yet further embodiments, the wearable device 105 may be configured to determine a context in which the vital signs are being determined. For example, a context may include, without limitation, an area of the body of the subject 145, or characteristics specific to the subject 145, from which signals are measured by the vital signs monitoring assembly. For example, the wearable device 105 may be configured to determine whether the sensor is receiving signals reflected from a specific body part of the subject 145 such as, without limitation, the hands, feet, arms, legs, wrists, elbows, neck, chest, back, or head. In some further examples, the wearable device 105 may be configured to determine further characteristics, such as height, weight, medical conditions, or other information that may affect the resultant signal. Accordingly, in some embodiments, context beyond skin tone level may be determined by the wearable device 105 based on inputs from the one or more sensors, to determine characteristics specific to the subject 145. Alternatively, the wearable device 105 may be deployed with an initial configuration based on the type of device of the wearable device. For example, a wearable device such as a headband may be configured to include algorithms for determining vital signals based on a reflected signal from a forehead or scalp, whereas a wristband device may be configured to determine vital signals based on reflected signals from a wrist or hand.

In further embodiments, the wearable device 105 may be configured to adjust the resultant signal it receives automatically, as described in further detail in the '463 patent. For example, the wearable device 105 may be configured to perform a calibration process. In some embodiments, the wearable device 105 may further include a calibration circuit for calibrating to its environment. Accordingly, in various embodiments, the wearable device 105 may be configured to adjust its physiological measurements based on the determination of the subject's skin tone level and other context of the wearable device 105.

In some embodiments, the wearable device 105 may be coupled to a network 150. The wearable device 105 may be configured to communicate with various remote devices, such as the provider system 140 or the user device 145. Accordingly, in some embodiments, the wearable device 105 may further include a communications subsystem, configured to communicate with remote devices, both directly or via network 150. Accordingly, in some embodiments, the provider system 140 may be a computer system associated with a healthcare provider. By maintaining communication with the wearable device 105, a healthcare provider may be able to monitor the subject 145 via the wearable device 105. Alternatively, the subject 145 may decide to contact their healthcare provider and provide information from the wearable device to the provider system 140. Similarly, data from the wearable device 105 may be accessed by a user, such as the subject 145, via user device 160 through a direct connection (e.g., a Bluetooth, or other wireless or wired connection), or via the network 150.

FIG. 2 illustrates a method 200 for determining a white SpO2 based on a polychromatic light source, as related to system 100 of FIG. 1, in accordance with at least one embodiment described herein. While in various embodiments, processes may be described in a certain order for purposes of illustration, it should be appreciated that certain processes may be reordered and/or omitted within the scope of various embodiments. Moreover, while the methods described can be implemented by (and, in some cases, were described with respect to) the system 100 of FIG. 1 (or components thereof), these methods may also be implemented using any suitable hardware implementation. Similarly, while the system 100 of FIG. 1 (and/or components thereof) can operate according to the methods described and/or the techniques described in the '463 patent (e.g., by executing instructions embodied on a computer readable medium), the system 100 may also operate according to other modes of operation and/or perform other suitable procedures.

In various embodiments, the method 200 begins, at block 205, by setting an equal gain for A and B channels of one or more light sensors. In various embodiments, the one or more light sensors include photodetectors and/or a spectral sensor as previously described. In some embodiments, the spectral sensor may be configured to measure spectral power distribution and/or intensity of light at one or more wavelengths. In some embodiments, each of the A and B channels may be associated with spectral power and/or intensity at a respective wavelength and/or range of wavelengths.

At block 210, A and B channel data is acquired using a polychromatic light source. In some embodiments, A and B channel data may be gathered by illuminating the subject with the polychromatic light source one or more times. For example, in some embodiments, the polychromatic light source may be configured to be turned on and off at 30 times a second. Accordingly, the polychromatic light source may be strobed and/or pulsed at 30 Hz.

The method 200 continues, at block 215, by determining a ratio of the two signals as R=100*B/A. The ratio may be indicative of a baseline measurement of the average spectral absorption and changes in spectral absorption of the subject tissue during an arterial pulse. At decision block 220, the method 200 continues by determining whether the sensor is in contact with the body of the subject. In some embodiments, this may be determined based on the calculated ratio R. If R is less than or equal to 1, then it may be determined, by the wearable device, whether one or more of the polychromatic light source, spectral sensor, or the wearable device itself is in contact with the body of the subject. If it is determined that there is no body contact, at block 225, a no contact flag may be triggered by the wearable device. The method 200 may then return to block 205, where gain for both A and B channel data is reset to be equal. If it is determined that body contact is present, the method 200 continues, at block 230, to find a minimum peak signal corresponding to an arterial pulse. At decision block 235, it is determined whether a minimum peak signal has occurred. If it is determined that a minimum peak has not occurred (e.g., an arterial pulse has not been detected or has not yet occurred), the method 200 returns to block 205, and gain for both A and B channels is reset to be equal, and A and B channel data is re-acquired (block 210) and the ratio R is recalculated (block 215).

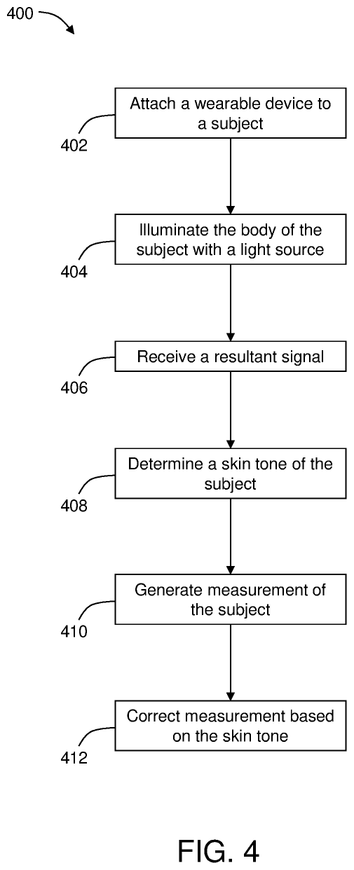

If, at decision block 235, a minimum peak is determined to have occurred, the method 200 continues, at block 240, by sampling and holding the ratio R. Using the sampled and held R, a moving average of the previous 25 sampled and held R values will be updated. At block 245, a white (e.g., polychromatic) light determined SpO2 value is looked up in a lookup table (such as the lookup table of FIG. 4), based on one or more of the sampled and held R value, or the moving average of the previous 25 sampled and held R values.

At decision block 250, it is determined whether a motion flag has been set. In various embodiments, the wearable device may include a motion sensor configured to determine whether the subject has moved. If movement has been detected (e.g., a motion flag set), at block 255, SpO2 values derived from monochromatic light sources, such as red and/or IR LEDs, will be substituted with white SpO2 values determined using the polychromatic light source. In various embodiments, movement of the subject may make it impossible to accurately calculate SpO2 of the subject based on monochromatic light sources. Accordingly, if a motion event occurs, the wearable device will rely on a polychromatic light source derived SpO2 value (e.g., white SpO2). If a motion flag has not been set, the method 200 returns to block 205, in which gain for channels A and B are reset to be equal.

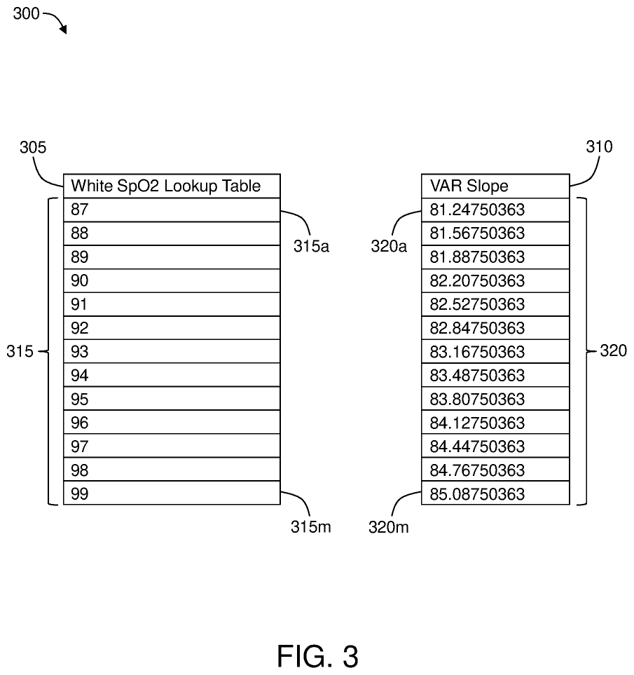

FIG. 3 illustrates lookup tables 300 for obtaining vital signs measurements in a wearable vital signs monitor, in accordance with at least one embodiment described herein. The lookup tables may include a white SpO2 lookup table 305 including a plurality of blood oxygen saturation values 315, and a VAR slope table 310 including a plurality of R values 320. Accordingly, a first R value 320a, corresponding to an R value of 81.24750363 may correspond to a first SpO2 value 315a of 87% blood oxygen saturation. A 13th R value 320m of 85.08750363 may correspond to the 13th SpO2 value 315m of 99% blood oxygen saturation. Accordingly each respective entry of the VAR slope table 310 may correspond to a respective entry of the white SpO2 lookup table 305.

Accordingly, SpO2 levels, derived from the polychromatic light source using the ratio R between A and B channels may be determined based on the lookup tables 300 as described above. In further embodiments, different lookup tables and lookup table values may be utilized based on a skin tone level and/or other context for the wearable device (e.g., an area of the body, or a characteristic of the subject). Accordingly, while embodiments of the wearable device may be configured to utilize the lookup tables 300, it is to be appreciated that in further embodiments, the wearable device may be configured to modify and/or use different lookup tables. In some embodiments, one or more lookup tables may be stored by the wearable device on, for example, an on-board computer or storage device. In some embodiments, the one or more lookup tables may be stored on a remote system, such as a separate, dedicated signal processing computer, server, database, or other remotely located device. Accordingly, the storage location of the lookup table may vary in different embodiments.

Wearable vital signs monitors as described herein may further include skin tone lookup tables that correlate spectral sensor signal values in response to illumination by polychromatic light with skin tone levels. The wearable vital signs monitor may use such a skin tone lookup table to determine the skin tone level of a subject based on one or more resultant signals from the polychromatic light illumination.

FIG. 4 is a method 400 for utilizing the wearable device, according to at least one embodiment described herein. While in various embodiments, processes may be described in a certain order for purposes of illustration, it should be appreciated that certain processes may be reordered and/or omitted within the scope of various embodiments. Moreover, while the methods described can be implemented by (and, in some cases, were described with respect to) the system 100 of FIG. 1 (or components thereof), these methods may also be implemented using any suitable hardware implementation. Similarly, while the system 100 of FIG. 1 (and/or components thereof) can operate according to the methods described and/or the techniques described in the '463 patent (e.g., by executing instructions embodied on a computer readable medium), the system 100 may also operate according to other modes of operation and/or perform other suitable procedures.

At block 402, the method 400 includes attaching the wearable device to a subject. For example, the wearable device may be positioned such that one or more of the polychromatic light source, spectral sensor, and/or one or more other sensors are operably coupled to the subject. In some embodiments, the wearable device may be positioned to be in contact with at least one of a wrist, palm, back of a hand, arm, chest, forehead, or earlobe of the subject.

At block 404, the method 400 includes illuminating the perfused tissue of the body of the subject with the polychromatic light source. In some embodiments, perfused tissue may include any tissue of the subject through which blood may be flowing. Illuminating the perfused tissue may include transmitting, directing, or otherwise providing light, from the polychromatic light source, to the surface of the perfused tissue, or alternatively, through the perfused tissue. The light may include light in a broad spectral range. In some embodiments, the broad spectral range spans at least 100 nanometers in the visible spectrum.

At block 406, the method 400 includes receiving a resultant signal by a spectral sensor of the wearable device. As previously described with respect to FIG. 1, the resultant signal may be a signal reflected by and/or received through the perfused tissue. The light source may be located adjacent to and facing a same direction as the spectral sensor, in which case the resultant signal may be a reflected signal. Alternatively, the light source may be located spaced apart from and facing the spectral sensor, in which case the resultant signal may be a transmitted signal. The spectral sensor that receives the resultant signal may include two or more detectors with different spectral response properties.

At block 408, the method 400 includes determining a skin tone level of the subject from or based on the received resultant signal. In embodiments in which the spectral sensor includes two or more detectors with different spectral response properties, receiving the resultant signal from the body of the subject at block 406 may include receiving the resultant signal at a first detector of the two or more detectors and at a second detector of the two or more detectors. Further, determining the skin tone of the subject from the received resultant signal at block 408 may include determining the skin tone of the subject from a first signal generated by the first detector responsive to receiving the resultant signal at the first detector and from a second signal generated by the second detector responsive to receiving the resultant signal at the second detector. Determining skin tone from the first and second signals may include performing signal processing on the first and second signals to accurately determine signal levels of the first and second signals. Determining the skin tone of the subject may further include comparing the signal levels to correlation data stored in a skin tone lookup table, the correlation data correlating signal levels to skin tone.

At block 410, the method 400 includes generating a physiological measurement of the subject based on a received resultant signal. The physiological measurement may include SpO2 or other physiological measurements described herein. The resultant signal may include the resultant signal from illuminating the subject with the polychromatic or other broad spectrum light source, the resultant signal from illuminating the subject with one or more monochromatic light sources (e.g., red and IR) of the wearable device, or a combination thereof.

At block 412, the method 400 includes correcting the physiological measurement based on the skin tone. Correcting the physiological measurement based on the skin tone may include accessing a specific lookup table or set of lookup tables (such as the lookup tables 300 of FIG. 3) corresponding to the determined skin tone to determine a skin-tone corrected physiological measurement. Alternatively or additionally, correcting the physiological measurement based on the determined skin tone may include modifying values of the specific lookup table or set of lookup tables (such as the lookup tables 300 of FIG. 3) according to a skin-tone-specific algorithm to determine the skin-tone corrected physiological measurement.

Some embodiments may include building skin tone lookup tables or other data structures from which skin tone level may be determined from resultant signals under polychromatic light illumination. This may be done by taking skin tone measurements across a population of subjects using a standard measurement technique and using wearable devices as described herein and then correlating the skin tone measurements generating using the wearable devices described herein with the skin tone measurements generated using the standard measurement technique.

Alternatively or additionally, embodiments herein may include building the skin-tone-specific lookup table (or set of lookup tables) or skin-tone-specific algorithms to apply to a single lookup table (or a single set of lookup tables) to generate skin-tone corrected physiological measurements. This may be done by, e.g., (1) taking physiological measurements across a population of subjects using a standard measurement technique unaffected by skin tone, (2) taking physiological measurements across the population using wearable devices as described herein, (3) taking skin tone measurements across the population using wearable devices as described herein, and then determining from the foregoing measurements how skin tone affects the physiological measurements using the wearable devices as described herein. This information may be used to build the skin-tone-specific lookup table (or set of lookup tables) or the skin-tone-specific algorithms to apply to a single lookup table (or single set of lookup tables) to generate skin-tone corrected physiological measurements.

In some embodiments, one or more lookup tables may be stored by the wearable device on, for example, an on-board computer or storage device. In some embodiments, the one or more lookup tables may be stored on a remote system, such as a separate, dedicated signal processing computer, server, database, or other remotely located device. Accordingly, the storage location of the lookup table may vary in different embodiments.

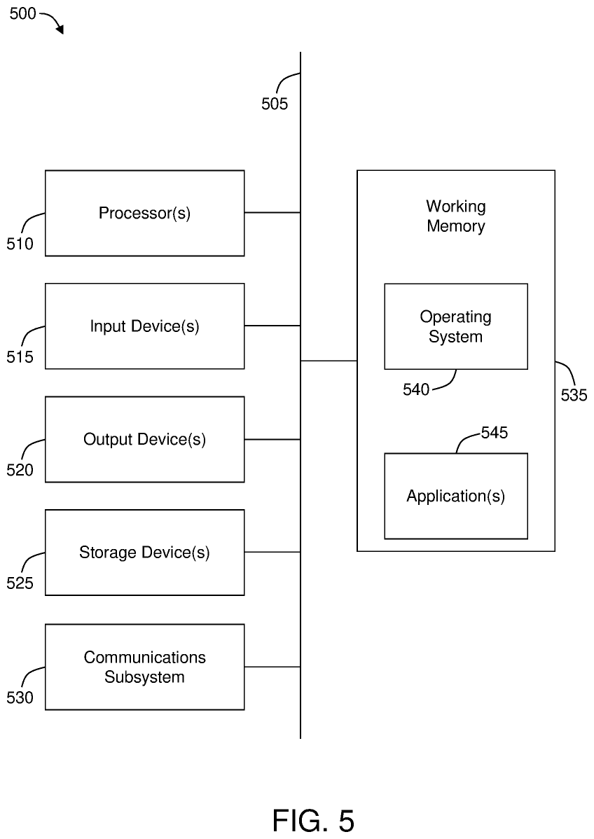

FIG. 5 is a schematic illustration of one embodiment of a computer system 500 configured to perform the methods provided by various other embodiments, as described herein. It should be noted that FIG. 5 is meant only to provide a generalized illustration of various components, of which one or more (or none) of each may be utilized as appropriate. FIG. 5, therefore, broadly illustrates how individual system elements may be implemented in a relatively separated or relatively more integrated manner.

The computer system 500 is shown comprising hardware elements that can be electrically coupled via a bus 505 (or may otherwise be in communication, as appropriate). The hardware elements may include one or more processors 510, including without limitation one or more general-purpose processors and/or one or more special-purpose processors (such as digital signal processing chips, graphics acceleration processors, and/or the like); one or more input device(s) 515, which may include, without limitation, one or more sensors, touchscreens, microphones, keyboards, computer mice, cameras, and other interface devices; one or more output devices 520, which may include, without limitation, a display, lights (e.g., LEDs), speakers, other indicators such as a vibrating element; one or more storage devices 525, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable and/or the like. Such storage devices may be configured to implement any appropriate data stores, including without limitation, various file systems, database structures, and/or the like.

The computer system 500 might also include a communications subsystem 530, which can include without limitation a modem, a network card (wireless or wired), an IR communication device, a wireless communication device and/or chipset (such as a Bluetooth™ device, an 802.11 device, a Wife device, a WiMax device, a WWAN device, cellular communication facilities, etc.), and/or the like. The communications subsystem 530 may permit data to be exchanged with a network (such as the network described below, to name one example), with other computer systems, and/or with any other devices described herein. In many embodiments, the computer system 500 will further comprise a working memory 535, which can include a RAM or ROM device, as described above.

The computer system 500 also may comprise software elements, shown as being currently located within the working memory 535, including an operating system 540, device drivers, executable libraries, and/or other code, such as one or more application programs 545, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and/or provided in an installation package, such that the storage medium can be used to program, configure and/or adapt a general purpose computer with the instructions/code stored thereon. These instructions might take the form of executable code, which is executable by the computer system 500 and/or might take the form of source and/or installable code, which, upon compilation and/or installation on the computer system 500 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer system 500) to perform and/or control methods in accordance with various embodiments of the invention. According to a set of embodiments, some or all of the procedures of such methods are performed and/or controlled by the computer system 500 in response to processor 510 executing one or more sequences of one or more instructions (which might be incorporated into the operating system 540 and/or other code, such as an application program 545) contained in the working memory 535. Such instructions may be read into the working memory 535 from another computer readable medium, such as one or more of the storage device(s) 525. Merely by way of example, execution of the sequences of instructions contained in the working memory 535 might cause the processor(s) 510 to perform or control performance of one or more procedures of the methods described herein.

The terms “machine readable medium” and “computer readable medium,” as used herein, refer to any medium that participates in providing data that causes a machine to operation in a specific fashion. In an embodiment implemented using the computer system 500, various computer readable media might be involved in providing instructions/code to processor(s) 510 for execution and/or might be used to store and/or carry such instructions/code (e.g., as signals). In many implementations, a computer readable medium is a non-transitory, physical and/or tangible storage medium. Such a medium may take many forms, including but not limited to, non-volatile media, volatile media, and transmission media. Non-volatile media includes, for example, optical and/or magnetic disks, such as the storage device(s) 535. Volatile media includes, without limitation, dynamic memory, such as the working memory 545. Transmission media includes, without limitation, coaxial cables, copper wire and fiber optics, including the wires that comprise the bus 505, as well as the various components of the communication subsystem 530 (and/or the media by which the communications subsystem 530 provides communication with other devices). Hence, transmission media can also take the form of waves (including without limitation radio, acoustic and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 500. These signals, which might be in the form of electromagnetic signals, acoustic signals, optical signals and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc. carried by the signals) to the working memory 535, from which the processor(s) 510 retrieves and executes the instructions. The instructions received by the working memory 535 may optionally be stored on a storage device 525 either before or after execution by the processor(s) 510.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An optical vital signs monitor, comprising:
a light source;
a spectral sensor;
a processor; and
a non-transitory computer readable medium comprising instructions executable by the processor to perform or control performance of operations comprising:
illuminating, via the light source, a body of a subject;
receiving, via the spectral sensor, a resultant signal from the body of the subject; and
determining a skin tone of the subject from the received resultant signal.

2. The optical vital signs monitor of claim 1, wherein the resultant signal comprises illumination radiation transmitted through the subject or reflected from the subject.

3. The optical vital signs monitor of claim 1, wherein the light source is configured to emit light in a broad spectral range.

4. The optical vital signs monitor of claim 3, wherein the broad spectral range spans at least 100 nanometers in the visible spectrum.

5. The optical vital signs monitor of claim 3, further comprising a second light source and a third light source, the second light source configured to emit radiation in the red spectral range and the third light source configured to emit radiation in the infrared spectral range.

6. The optical vital signs monitor of claim 1, wherein the light source:
is located adjacent to and facing a same direction as the spectral sensor; or
is located spaced apart from and facing the spectral sensor.

7. The optical vital signs monitor of claim 1, wherein the spectral sensor comprises two or more detectors with different spectral response properties.

8. The optical vital signs monitor of claim 7, wherein:
receiving the resultant signal from the body of the subject includes receiving the resultant signal at a first detector of the two or more detectors and at a second detector of the two or more detectors; and
determining the skin tone of the subject from the received resultant signal comprises determining the skin tone of the subject from a first signal generated by the first detector responsive to receiving the resultant signal at the first detector and from a second signal generated by the second detector responsive to receiving the resultant signal at the second detector.

9. The optical vital signs monitor of claim 8, wherein determining the skin tone of the subject from the first and second signals comprises performing signal processing on the first and second signals to accurately determine signal levels of the first and second signals.

10. The optical vital signs monitor of claim 9, further comprising a lookup table stored in the non-transitory computer readable medium, wherein determining the skin tone of the subject further comprises comparing the signal levels to correlation data stored in the lookup table, the correlation data correlating signal levels to skin tone.

11. The optical vital signs monitor of claim 10, the operations further comprising:
generating a physiological measurement of the subject based on the received resultant signal; and
correcting the physiological measurement based on the determined skin tone.

12. The optical vital signs monitor of claim 11, wherein the physiological measurement comprises peripheral oxygen saturation (SpO₂) of the subject.

13. A method, comprising:
illuminating, via a light source of an optical vital signs monitor, a body of a subject;
receiving, via a spectral sensor of the optical vital signs monitor, a resultant signal from the body of the subject; and
determining a skin tone of the subject from the received resultant signal.

14. The method of claim 13, wherein receiving the resultant signal comprises receiving illumination radiation transmitted through the subject or reflected from the subject.

15. The method of claim 13, wherein illuminating the body of the subject comprises illuminating the body of the subject with light in a broad spectral range.

16. The method of claim 15, wherein the broad spectral range spans at least 100 nanometers in the visible spectrum.

17. The method of claim 15, further comprising separately illuminating the body of the subject with red light and infrared light.

18. The method of claim 13, wherein:
the spectral sensor comprises two or more detectors with different spectral response properties;
receiving the resultant signal from the body of the subject includes receiving the resultant signal at a first detector of the two or more detectors and at a second detector of the two or more detectors; and
determining the skin tone of the subject from the received resultant signal comprises determining the skin tone of the subject from a first signal generated by the first detector responsive to receiving the resultant signal at the first detector and from a second signal generated by the second detector responsive to receiving the resultant signal at the second detector.

19. The method of claim 18, wherein determining the skin tone of the subject from the first and second signals comprises performing signal processing on the first and second signals to accurately determine signal levels of the first and second signals.

20. The method of claim 19, wherein determining the skin tone of the subject further comprises comparing the signal levels to correlation data stored in a lookup table on the optical vital signs monitor, the correlation data correlating signal levels to skin tone.

21. The method of claim 10, further comprising:
generating a physiological measurement of the subject based on the received resultant signal; and
correcting the physiological measurement based on the determined skin tone.

22. The method of claim 21, wherein the physiological measurement comprises peripheral oxygen saturation (SpO₂) of the subject.
